(19) United States
(12) Patent Application Publication
(10) Pub. No.: US 2007/0129750 A1
(43) Pub. Date: Jun. 7, 2007

(54) Catheter balloon device with internal guidewire lumen and method of formation

(75) Inventor: Travis Yribarren, San Mateo, CA (US)

Correspondence Address: BEYER WEAVER & THOMAS LLP, P.O. BOX 70250, OAKLAND, CA, 94612-0250, US

(73) Assignee: Abbott Laboratories, Redwood City, CA (US)

(21) Appl. No.: 11/634,596

(22) Filed: Dec. 5, 2006

Related U.S. Application Data
(60) Provisional application No. 60/742,765, filed on Dec. 5, 2005.

(51) Int. Cl. A61M 29/00

(52) U.S. Cl. 606/194

(57) ABSTRACT

An inflatable working element is provided for a catheter device, the catheter device having an elongated flexible tubular shaft containing an inflation lumen therethrough. The working element includes a hollow bladder device selectively inflatable from a first condition to an expanded second condition. The bladder device defines an inflation passage extending from a proximal end opening to a distal end opening thereof. The proximal end opening is configured to cooperate with a distal portion of the elongated shaft such the inflation passage is in access communication with the shaft inflation lumen. The bladder device further includes an inverted tubular arm member configured to be disposed in the inflation passage. The tubular arm member includes a first end port accessible through the proximal end opening of the bladder device and an opposed second end port integrally formed with, and terminating at, a sidewall of the bladder device.

### RELATED APPLICATION DATA

The present application claims priority under 35 U.S.C. §119 to U.S. Provisional Application Ser. No. 60/742,765, naming Yribarren as inventor, filed Dec. 5, 2005, and entitled CATHETER BALLOON DEVICE WITH INTERNAL GUIDEWIRE LUMEN AND METHOD OF FORMATION, the entirety of which is incorporated herein by reference for all purposes.

### FIELD OF THE INVENTION

The present invention relates generally to catheters, and more particularly, relates to expandable or inflatable working elements for the catheters that are designed to permit a guidewire or other structure to be fed laterally therefrom. Such arrangements are believed to be particularly useful in devices that are intended for use in the vicinity of vessel bifurcations.

### BACKGROUND OF THE INVENTION

Inflatable catheters are applied in a very wide variety of vascular medical procedures, such as, for instance, angioplasty and stenting. These catheters are generally relatively long and flexible, and include a selectively inflatable or expandable working element proximate a distal end thereof. Typical applications of these inflatable catheters include balloon dilation and/or stent deployment. During vascular placement, the inflatable working element of the delivery catheter may need to be negotiated through a relatively tortuous vessel. Since it can be difficult to steer many types of catheters, guidewires are often deployed to guide and position the catheters through the vessel.

Guidewires, on the other hand, are usually formed from a very small diameter wire having a flexible tip that may be bent (typically pre-bent) by a physician. These pre-bent shapes facilitate “steering” of the guidewire to a desired location through a potentially tortuous path such as the vasculature.

In some applications the targeted region of a vessel may be at a location where the vessel bifurcates. For example, in cases where atherosclerotic plaque has developed in the region of a vascular vessel bifurcation, it may be desirable to perform a therapeutic treatment on the affected vessels. In some applications, it may be desirable to stent one or both branches of a vessel bifurcation. In other applications, it may be desirable to perform an operation such as angioplasty or atherectomy in one branch, while stenting the other branch.

One common procedure for intraluminally implanting a stent is to first open the relevant region of the vessel through balloon dilation (via a balloon catheter or the like) and then place the stent in a position that bridges the treated portion of the vessel in order to prevent elastic recoil and restenosis of that segment. The angioplasty of the bifurcation lesion has traditionally been performed using the “kissing” balloon technique where two guidewires and two balloons are inserted, one into the main branch and the other into the side branch. Stent placement in this situation requires the removal of the guidewire from the side branch and, subsequently, reinsertion of a guidewire through the stent struts. This is followed by the insertion of a balloon through the struts of the stent along the side branch guidewire. The first removal of the guidewire poses the risk of vessel dissection.

In these situations where the main or side branch has been stented, sometimes the scaffolding of the stent is positioned directly over the bifurcation, and prevents or significantly increases the difficulty of passing the side branch guidewire through the stent structure for treating the other vessel. This is due, in-part, to the fact the diameter of the side branch is often much smaller than that of the main branch. More significantly, the angle between the two branches can be relatively large. In most bifurcations the vessels branch at a bifurcation angle of less than 60 degrees, but there are also vessel bifurcations in which the bifurcation angles are in the range of 60-90 degrees and sometimes even greater. Especially in cases where the bifurcation angle is greater than 60 degrees it can be difficult to pass a stent after implantation.

Moreover, when treating a bifurcation using this technique, it is often important for the physician to be able to easily and immediately access the second vessel after deployment of the stent. As mentioned, in the current systems, after the stent is deployed, the side branch guidewire must be removed to enable access to the side branch through the deployed stent before reinsertion of the side branch guidewire can be achieved. It is also possible for the main branch guidewire to be placed in the side branch before removing and re-inserting the side branch guidewire.

Depending on the nature of the stenosis it might be possible that plaque shifting occurs during the treatment. This can occur when one of the vessels is dilated with a balloon or a stent is placed. Plaque shifting (which is sometimes referred to as the “snowplow effect” may then occlude (or partially occlude) the other vessel. To re-open the vessel, first a guidewire has to be placed in the second vessel. Depending on the lesion, the physician might decide to dilate the second vessel with a balloon catheter or place another stent.

Although there are currently a number of devices that are designed for use in the region of vessel bifurcations, there are continuing efforts to provide improved mechanisms for accessing a side branch after stent deployment. Hence, it would be desirable to provide a catheter with an inflatable working element that allowed the passage of a guidewire through the element wall to permit access to the non-treated vessel of a bifurcation.

### SUMMARY OF THE INVENTION

The present invention provides a unitary inflatable working element for use with a delivery catheter device or the like suitable for treating a vessel bifurcation. Typically, the catheter device includes an elongated flexible tubular member containing at least an inflation lumen and a main guidewire lumen. The working element includes a hollow balloon or bladder device selectively inflatable from a first condition to an expanded second condition. The bladder device defines an inflation passage extending from a proximal end opening to a distal end opening thereof. The proximal end opening is configured to cooperate with a distal portion of the elongated tubular member such the inflation passage is in flow communication with the shaft inflation lumen. The working element further includes a tubular arm member configured to be disposed in the inflation passage. The arm member includes first portion defining a first end port accessible through the proximal end opening of the bladder device and an opposed second portion, defining a second end port, and being coupled to, and terminating at, a sidewall of the bladder device in a fluid-tight manner.

Accordingly, a secondary guidewire lumen is formed from the tubular arm member that is accessible through the inflation passage of the working element and which exits the sidewall of the bladder device. Moreover, the secondary guidewire lumen is fully accessible and operational while the bladder device is in its inflated condition. This enables the positioning of both a main guidewire and of a second guidewire through the secondary guidewire lumen without disturbing the operation of the working element. Such access is extremely advantageous to enable access to a side branch of a bifurcated vessel.

In one specific embodiment, the tubular arm member is integrally formed with the bladder device at the second end port. The bladder device and the tubular arm member may even comprise a one-piece formed working element.

Another specific configuration tapers the tubular arm radially inward from the second portion thereof toward the first portion thereof. A longitudinal axis of the tubular arm, in a natural state, and the longitudinal axis of the bladder device are substantially contained in a same plane.

In still another embodiment, the longitudinal axis of the tubular arm at the second portion thereof, in the natural state, is oriented at an angle in the range of about 20° to about 90° relative to the longitudinal axis of the bladder device.

In another aspect of the present invention, method of fabricating an inflatable balloon element is disclosed for a catheter device suitable for treating a vessel bifurcation. The method includes forming a unitary balloon element having a hollow bladder portion defining an inflation passage extending from a proximal end opening to a distal end opening thereof. The body portion includes a flexible tubular arm member having a first end defining a first end port directed generally radially away from the body portion of the balloon element, and an opposed second end integrally formed in a sidewall of the body portion. The second end defines a second end port that terminates at the body portion inflation passage such that the second end port is in direct access communication with the inflation passage. The method further includes inverting one of the tubular arm member and the hollow bladder portion inside out such that the arm member is now disposed in the inflation passage, and having the first end port accessible through the proximal end opening of the bladder device and the opposed second end terminating at the sidewall in a manner such that the second end port is out of direct access communication with the inflation passage.

In one specific embodiment, the method molding the hollow bladder portion about a balloon mold device as a one-piece element configured in a predetermined shape to form a shell body. Such a formation may include molding the tubular arm member about a core pin that is removably mounted into a side of the first mold device. The core pin is then removed from the first mold device, leaving the tubular arm.

In still another specific approach, the forming a unitary balloon element further includes placing the shell body into a chamber of a mold shell having interior walls substantially preshaping the chamber into the desired final shape of the balloon element. Heat is applied to the chamber, and then the inflation passage of the balloon Element is inflated, expanding the hollow bladder against the interior walls of the mold shell.

In still another specific approach, the forming a unitary balloon element further includes placing the shell body into a chamber of a mold shell having interior walls substantially preshaping the chamber into the desired final shape of the balloon element. Heat is applied to the chamber, and then the inflation passage of the balloon element is inflated, expanding the hollow bladder against the interior walls of the mold shell.

Prior to applying heat, in another specific embodiment, the method include inserting the tubular arm into a side port of the interior walls of the mold shell that is formed and dimensioned for axial receipt of the tubular arm therein.

In yet another specific aspect of the present invention, a method is provided for fabricating a catheter device suitable for treating a vessel bifurcation. The method includes The method includes forming a unitary balloon element having a hollow bladder portion defining an inflation passage extending from a proximal end opening to a distal end opening thereof. The body portion includes a flexible tubular arm member having a first end defining a first end port directed generally radially away from the body portion of the balloon element, and an opposed second end integrally formed in a sidewall of the body portion. The second end defines a second end port that terminates at the body portion inflation passage such that the second end port is in direct access communication with the inflation passage. The method further includes inverting one of the tubular arm member and the hollow bladder portion inside out such that the arm member is now disposed in the inflation passage, and having the first end port accessible through the proximal end opening of the bladder device and the opposed second end terminating at the sidewall in a manner such that the second end port is out of direct access communication with the inflation passage. The proximal end of the balloon element is then mounted to a distal portion of an elongated shaft of the catheter device such that the inflation passage of the balloon element is in flow communication with an inflation lumen of the catheter elongated shaft.

In one particular configuration, the forming a unitary balloon element includes selecting a length of the tubular arm such that the first end port extends through and terminates proximal to the proximal end opening of the hollow bladder portion. The mounting includes positioning the first end of the tubular arm through the proximal end opening of the hollow bladder portion, and between the distal portion of the elongated shaft and the hollow bladder portion.

In yet another specific embodiment, the forming a unitary balloon element includes selecting a length of the tubular arm such that the first end port terminates distal to the proximal end opening of the hollow bladder portion. The mounting includes coupling the first end port of the tubular arm in fluid-tight access communication with a guidewire lumen extending through the elongated shaft.

In still another aspect of the present invention, a catheter device is provided including an elongated flexible tubular shaft containing an inflation lumen and a main guidewire lumen therethrough. An inflatable working element is mounted to the flexible tubular shaft, and includes a hollow bladder portion selectively inflatable from a first condition to an expanded second condition. The bladder portion defines an inflation passage extending from a proximal end opening to a distal end opening thereof. The proximal end opening is configured to cooperate with a portion of the elongated shaft such that the inflation passage is in access communication with the shaft inflation lumen. The working element further includes a tubular arm member configured to be disposed in the inflation passage. A first end port of the arm member is accessible through the proximal end opening of the bladder portion and an opposed second end port integrally formed with, and terminating at, a sidewall of the bladder portion

In one specific embodiment, the tubular arm member is sized such that the first end port extends through and terminates proximal to the proximal end opening of the bladder device. The first end of the tubular arm, thus, extends through the proximal end opening of the hollow bladder portion, and between a distal portion of the elongated shaft and the hollow bladder portion.

In another specific configuration, the tubular arm is sized such that the first end port terminates distal to the proximal end opening of the bladder device within the inflation passage. Further, the elongated shaft includes a secondary guidewire lumen in fluid-tight access communication with first end port of the tubular arm.

The main guidewire lumen extends through a distal end of elongated shaft, the proximal end opening of the hollow bladder portion and the distal end opening thereof. In one particular embodiment, the secondary guidewire lumen and the main guidewire lumen are in access communication with one another.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawing, in which:

FIG. 1 is a fragmentary, side elevation view, in cross-section, of a catheter assembly constructed in accordance with the present invention, deployed in a bifurcated vessel.

FIG. 2 is a fragmentary, side elevation view, in cross-section, of the catheter assembly of FIG. 1, illustrating a tubular arm member or an inflatable working element.

FIG. 3 is a side elevation view, in cross-section, of the inflatable working element of FIG. 2

FIG. 4 is a fragmentary, side elevation view, in cross-section, of an alternative embodiment catheter assembly

FIG. 5 is a side elevation view of a balloon mold constructed in accordance with the present invention.

FIG. 6 is a side elevation view of an initial cylindrical shell body formed from the balloon mold of FIG. 5.

FIG. 7 is a side elevation view, in cross-section, of an inflatable working element being inverted through a tool device.

FIG. 8 is a fragmentary, side elevation view, in cross-section, of a mold shell with the initial cylindrical shell body of FIG. 6, before deformation to its final shape of FIG. 7.

### DETAILED DESCRIPTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

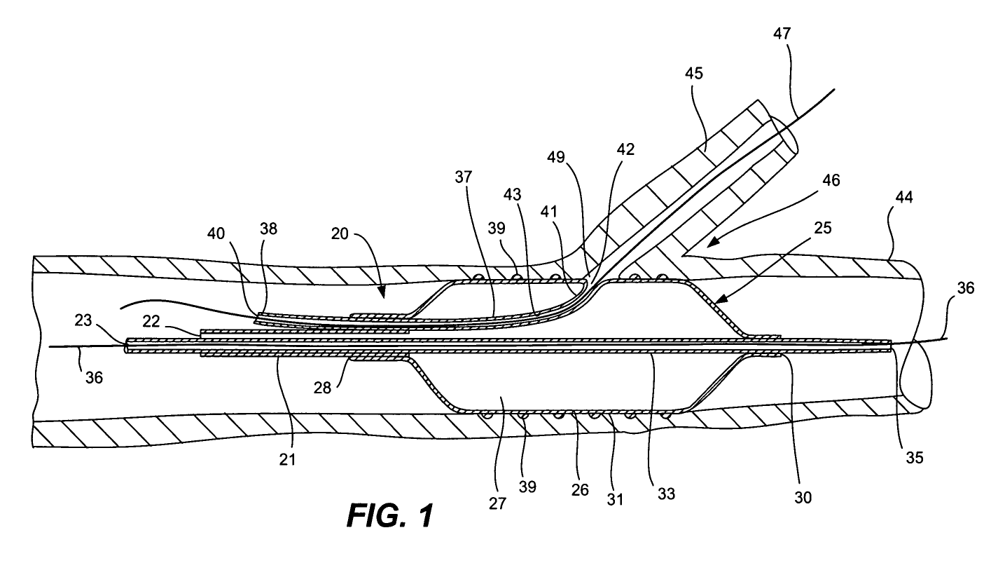

Referring now to FIGS. 1-3, a delivery catheter assembly, generally designated 20, is provided having an elongated tubular member or catheter shaft 21 that defines at least one inflation lumen 22 extending through the shaft. As will be appreciated by those familiar with the art, only the distal, working end of the catheter assembly 20 is shown in these figures for illustrative purposes. The length and size of the catheter shaft 21 will typically depend on its desired application and the proximal end (not shown) of the catheter would typically be outfitted with a suitable handle and ports, valves and other structures for controlling the working (distal) end of the catheter.

The delivery catheter assembly 20 is particularly suitable for deployment in vascular vessels including coronary vessels. However, in other embodiments, the catheter may be designed for insertion in any body vessel or tubular structure of the body. The flexible catheter shaft 21 may include any suitable number of lumens. In the illustrated embodiment, the lumens include at least a main guidewire lumen 23 and inflation (e.g., fluid supply) lumen 22.

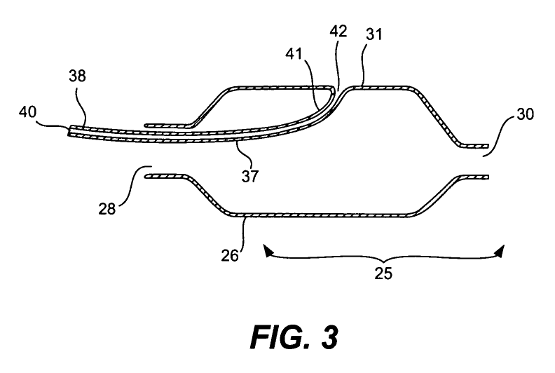

Mounted to a distal portion of the catheter shaft 21 is a unitary inflatable working element 25 of the catheter assembly 20. Briefly, while the unitary working element is preferably a one-piece unit, it may be constructed of an integration of multiple pieces as well. Referring back to FIG. 3, the working element 25 contains a substantially cylindrical hollow bladder portion 26 defining an inflation passage 27 extending longitudinally therethrough from a proximal end opening 28 to a distal end opening 30. The hollow bladder portion includes a substantially thin sidewall 31 with a substantially cylindrical exterior surface and an opposed interior surface. A proximal portion of the working element 25 tapers radially inward toward the proximal end opening 28, as does a distal portion of the working element 25 that tapers radially inward toward the distal end opening 30.

Similar to most conventional inflatable balloon catheter designs, the proximal end opening 28 and the distal end opening 30 are sized to mount and seal to respective portions of the elongated catheter shaft 21 of the catheter assembly, while the substantially thin and flexible central bladder portion 26 is configured for selective inflation from an unexpanded first condition to an expanded second condition (FIG. 1). Hence, the transverse cross-sectional dimension of the inflation passage 27, in the expanded second condition, is significantly greater than that of the inwardly tapered end portions of proximal end opening 28 and the distal end opening 30.

When the inflatable working element 25 is mounted to the flexible catheter shaft 21, the inflation lumen 22 of the catheter shaft is in flow communication with the inflation passage 27 of the working element 25. Accordingly, by operating the control systems at the proximal end of the catheter assembly, the central bladder portion 26 of the working element 25 can be selectively inflated from the first condition to the inflated second condition.

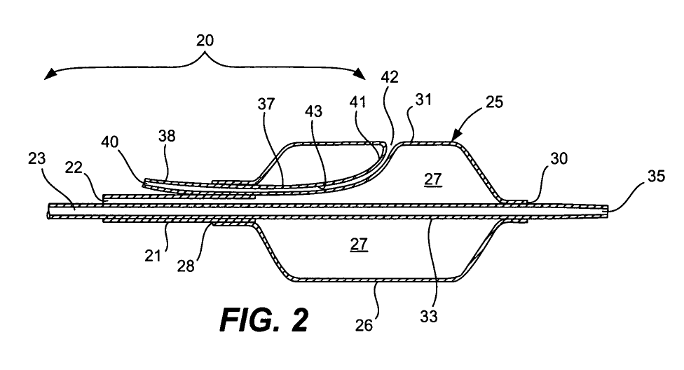

A distal tube portion or extension 33 of the flexible catheter shaft 21 extends through the inflation passage 27 of the inflatable working element 25, where a distal end of the catheter assembly 20 terminates just past the distal end opening 30 of the working element. As best shown in FIG. 2, the catheter shaft distal tube portion 33 extends longitudinally through the working element 25, and defines the distal portion of the main guidewire lumen 23 where it terminates at a distal port 35 at a distal end of the catheter shaft 21. Hence, a main guidewire 36 may extend through the main guidewire lumen 23 of the catheter assembly 20, and out through the distal port 35 of the catheter distal end. This passage enables the catheter shaft 21 to be advanced along the main guidewire 36 that is strategically disposed in a vessel.

FIG. 2 best shows that the distal tube portion 33 defines at least a portion of the main guidewire lumen 23. For example, the distal tube portion 33 may simply extend from the distal end of the main portion of the catheter shaft 21, having at least one side integral therewith as shown in FIG. 3. In another configuration, the tube portion 33 may actually be contained within and extend through at least a portion of a larger lumen of the catheter shaft in a manner independent of the shaft.

Referring back to FIG. 1, an interior wall defining the distal end opening 30 of the working element 25 is configured to seal around the corresponding outward facing surface of the distal tube portion 33 in a fluid-tight manner. Similarly, a fluid-tight seal is formed between an interior wall defining the working element proximal end opening 28 and the corresponding exterior facing surface of the catheter shaft 21 and/or at least a portion of the distal tube portion 33. Collectively, these two seals isolate the main guidewire lumen 23 from the inflation passage 27 of the working element 25, and permit inflation of the element.

In accordance with the present invention, the working element 25 includes an inverted tubular arm member 37 integrally formed with the hollow bladder portion 26. The tubular arm member 37 includes a first end 38 that defines a first end port 40, and an opposed second end 41, integrally formed and attached (e.g., molded, bonded or adhered) to the sidewall 31 of the hollow bladder portion 26, that defines a second end port 42. The tubular arm member 37 further includes a side lumen or secondary guidewire lumen 43 extending therethrough from the first end port 40 to the second end port 42.

In the inverted configuration of the tubular arm member 37, as shown FIG. 3, the tubular arm member 37 passes through the inflation passage 27 in the direction toward the proximal end opening 28. In one specific embodiment, the length of the arm member 37 is sufficient to position the first end 38 at least through and just beyond the proximal end opening 28 of the working element proximal portion. Accordingly, in the inverted state, the secondary guidewire lumen 43 is now essentially isolated from flow access to the inflation passage 27, and the second end port 42 is out of direct access communication with the working element inflation passage 27. Rather, the second end port 42 is in direct access communication with the exterior of the balloon or bladder portion 26 that enables access communication through the inflation lumen and out of the sidewall 31 of the bladder portion 26.

Both the main guidewire lumen 23 and the secondary guidewire lumen 43, hence, are formed that simultaneously permit passage through the inflation lumen 22 of the working element 25 without any disruption in operation thereof. In particular, the secondary guidewire lumen 43 provides easy access to a side branch vessel 45 of a bifurcated vessel 46 directly through the sidewall 31 of the working element bladder portion 26 (FIG. 1). This accessibility is possible even after inflation of the working element and deployment of a stent 39.

Once the catheter assembly 20 is manipulated along the main guidewire 36 until the working element 25 is at least partially disposed in the main vessel 44 of the bifurcated vessel 46, the second end port 42 is aligned with the opening 49 into the side branch vessel 45. Such alignment is maintained during inflation of the bladder portion 26 from the first condition to the inflated second condition, although constant alignment may not be necessary. Subsequently, a second guidewire 47 can be negotiated into secondary guidewire lumen 43 of the tubular arm member 37. Unlike the current systems, the working element 25 will not require deflation and removal thereof prior to guidance of the tip of the second guidewire therethrough. Hence, since the inflated working element 25 remains inflated in the second condition, the tip of the second guidewire can be confidently navigated and negotiated through the working element 25, via the secondary guidewire lumen 43 of the tubular arm member 37, and out of the bladder portion sidewall 31, via second end port 42. Once past this juncture, the tip will be guided through the scaffolding of the deployed primary stent 39 and into the side branch vessel 45 of the bifurcated vessel 46. Such deployment and alignment of the second guidewire 47 to the side branch vessel 45, accordingly, is substantially simplified.

As best viewed in FIGS. 2 and 3, the transverse cross-sectional dimension of the secondary guidewire lumen 43 of the tubular arm member 37 is substantially smaller then both that of the inflation passage 27 and of the proximal end opening 28 of the working element 25. In general, the working element proximal end opening 28 must be sized to receive at least the distal tube portion 33 of the catheter shaft 21 upon which it is sealed and mounted to. In turn, the catheter shaft 21 must be sized to accommodate at least the main guidewire lumen 23 and the inflation lumen 22.

In one particular configuration shown in FIGS. 1 and 2, the first end 38 of the tubular arm member 37 is positioned between the catheter shaft 21 and the proximal portion of the working element 25 that defines the proximal end opening 28. As illustrated, the first end 38 of the tubular arm member 37 must be of sufficient length to pass through the proximal end opening 28 where the first end port 40 is accessible. The interior wall defining the distal end opening 30 of the working element 25 is sealed against the exterior surface of the catheter shaft and against the tubular arm member to properly isolate the inflation passage of the bladder portion 26.

The first end 38 of the tubular arm member 37 may be mounted to a tube device or the like (not shown) that enables access to the secondary guidewire lumen 43 at a position more proximal to the operating end of the catheter assembly. Such tube device may be internal or external to the catheter shaft 21. This will ease advancement of the second guidewire to the working element 25. In another configuration, as shown in FIG. 1, the secondary guidewire lumen 43 may just be accessible at the first end port 40 of the tubular arm member 37 just proximal to the working element 25. In still another configuration, the secondary guidewire lumen 43 of the tubular arm member 37 may enter the catheter shaft 21 and exit through a welded transition as in RX technology more proximal to the operating end of the catheter assembly.

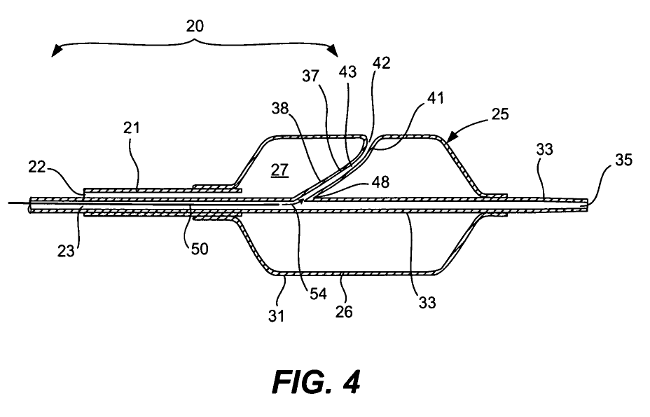

In either configuration, the second (side branch) guidewire 47 will seamlessly enter the secondary guidewire lumen 43 of the tubular arm member 37, via the first end 38. This permits the second guidewire 47 to pass through the inflation lumen 22 of an inflated working element 25, and out through the sidewall 31 thereof, via the tubular arm second end port 42, without requiring deflation or removal of the working element 25. In fact, as shown in FIGS. 4, this design enables access to both the main and secondary guidewires through the inflated catheter balloon or working element at the treatment area. As mentioned, this is very advantageous in that the catheter assembly 20 need not be subsequently realigned with the side branch vessel 45 in order to negotiate the second guidewire 47 into the side branch vessel 45, as the current designs require. Another benefit includes not needing to switch wires within the anatomy, thereby reducing the risk of dissection. It will be appreciated that the guidewire may also be back loaded into the side lumen at the distal end.

In another alternative configuration not shown, the first end 38 of the tubular arm member 37 may terminate distal to the proximal end opening 28 and may be sealably mounted to or communicate with structure contained internally within the catheter shaft 21 that defines a shaft secondary guidewire lumen. It will be appreciated, however, that such a sealed mount to this secondary guidewire structure of the catheter shaft could also be performed inside the working element inflation passage 27 as well. In fact, in the embodiment illustrated in FIG. 4, the first end 38 of the tubular arm member 37 can be configured to communicably intersect the main guidewire lumen 23 within the inflation passage 27 of the working element 25. This communication intersection 48 between the arm member secondary guidewire lumen 43 and the main guidewire lumen 23, which incidentally can also occur outside of the inflation passage 27, permits access communication with both lumens from a single main guidewire lumen 23 of the catheter shaft 21.

This arrangement is beneficial in that the entire treatment of the bifurcated vessel 46 may be performed using a single guidewire 50. For example, once the catheter is aligned and the working element 25 is inflated to deploy the stent 39, then the single guidewire 50 can be retracted until the distal tip thereof is just proximal to the communication intersection 48. Subsequently, the distal tip of the single guidewire 50 is oriented and navigated into and through the secondary guidewire lumen 43 (along the path of arrow 54 in FIG. 4). After passing through the second end port 42, the guidewire is passed through the stent scaffolding and into the side branch vessel 45.

After the working element 25 is deflated, the catheter shaft 21 can be withdrawn along the guidewire 50 while retaining the distal tip thereof in the side branch vessel 45. Subsequently, another catheter can be positioned along the same guidewire 50, and into the side branch vessel 45 to complete the procedure.

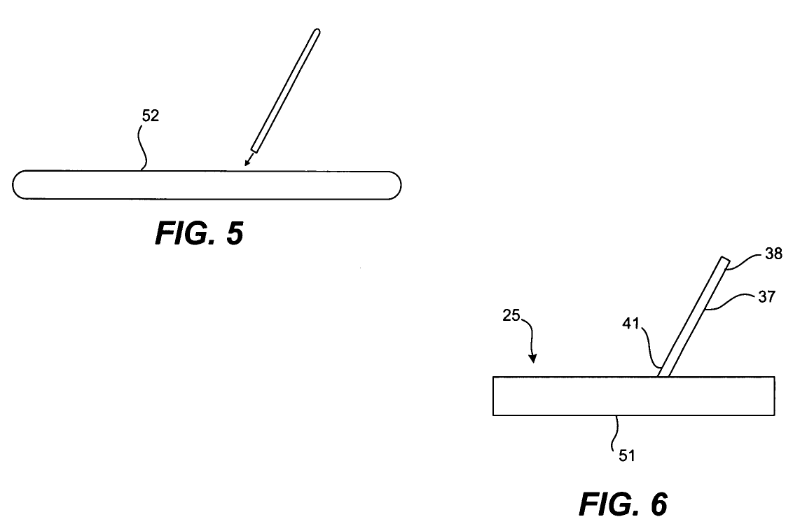

In accordance with another aspect, the present invention includes a technique of fabrication of the inflatable working element 25 with the tubular arm member 37. As best illustrated in FIGS. 5 and 6, in one embodiment, the working element 25 is initially formed using a balloon mold 52 having a substantially cylindrical shape. The balloon mold 52, as will be described, is applied using conventional injection molding, dip molding, blow molding, electrograft techniques, or bonding of individual tubings using welding or solvent bonding techniques, to fabricate a substantially cylindrical shell-shaped mold body containing the proximal end opening 28 and the distal end opening 30.

To form the tubular arm member 37, a removable core pin 53, having the desired diameter for the secondary guidewire lumen 43 (albeit inverted), is positioned into a hole (not shown) in the side of the balloon mold 52. The location and position of the core pin 53 is, of course, pre-selected so as to properly position the second end port 42 at the desired location relative bladder portion 26. Moreover, the angle and direction of the core pin 53 are pre-selected so as to properly angle and position the tubular arm member 37 in the desired direction. The removable core pin, for example, is preferably angled relative a longitudinal axis of the cylindrical shell body 51 in the range of about 20° to about 90°, and most preferably about 60°. It will be appreciated that smaller angles can aid in reducing the overall system profile, while larger angles can improve the ease of guidewire insertion and tracking. Further, while the core pin may be substantially linear and uniform in diameter, it will be appreciated that the core pin may also be curvilinear and/or non-uniform in diameter, as long as the core pin can be removed from the tubular arm member without jeopardizing the integrity thereof.

In accordance with the present invention, however, the tubular arm member 37 is integrally fabricated into the cylindrical shell body 51 in this configuration. This is performed by inserting a removable core pin 53, having the desired diameter for the secondary guidewire lumen 43, into a hole in the side of the balloon mold 52. The insertion of the core pin 53, of course, is to be at a pre-selected location so as to properly position the second end port 42 as the desired location. Further, the orientation of the core pin, relative to the balloon mold 52, is retained at a pre-selected angle and direction. The removable core pin, for example, is angled relative a longitudinal axis of the balloon mold 52 in the range of about 20° to about 90°, and most preferably about 60°. Further, while the core pin may be substantially linear and uniform in diameter, it will be appreciated that the core pin may also be curvilinear and/or non-uniform in diameter, as long as the core pin can be removed from the tubular arm member 37 without jeopardizing the integrity thereof.

After initial molding or electrografting of the cylindrical shell body 51 and the tubular arm member 37 using conventional molding techniques, the core pin 53 is removed from the cylindrical balloon mold 52, leaving the tubular arm member 37 in tact with the cylindrical shell body 51. Subsequently, the cylindrical shell body 51 is removed from of the balloon mold 52 leaving the initial cylindrical shape of the working element 25, as shown in FIG. 6.

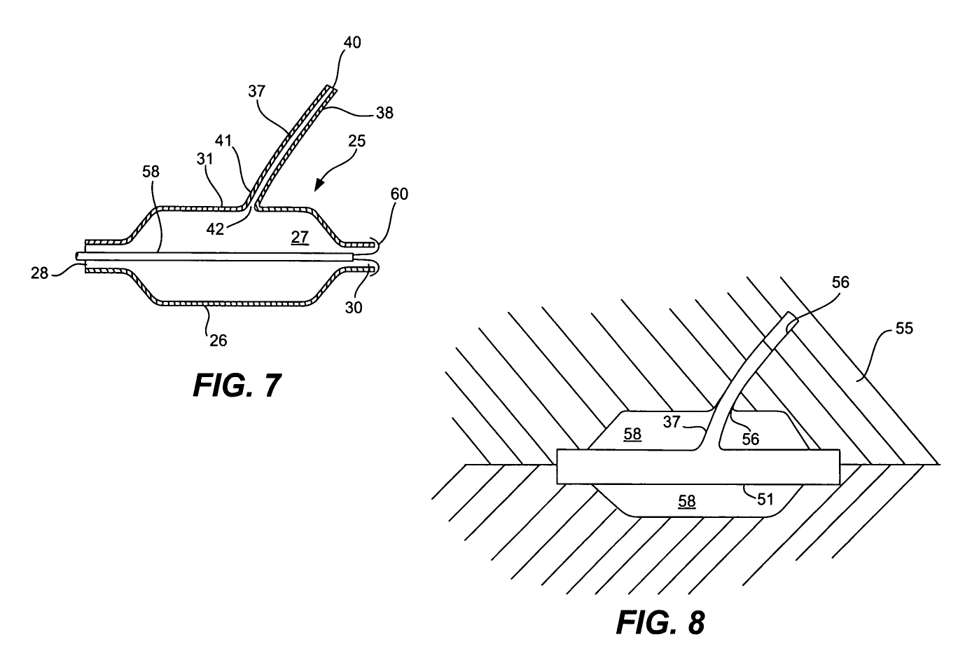

To function in accordance with the present invention, the tubular arm member 37 must be in an inverted configuration (FIG. 3) relative to the initial molded configuration of the cylindrical shell body 51 (FIG. 6). Briefly, it will be appreciated that the inverted state of the tubular arm member is a relative term in that, as will be described below, this configuration can accomplished by either inverting the tubular arm member 37 itself inside out or inverting the hollow bladder portion 26 inside out. For example, in the latter case, an elongated tool 58 or the like with a plurality of pronged ends 60 can be passed through the inflation passage 27 of the working element. Using the pronged ends 60, as shown FIG. 7, either the proximal end or the distal end of the working element 25 can be engaged and pulled longitudinally through the center thereof. Hence, in this technique, the bladder portion 26 is inverted rather than the much smaller diameter tubular arm member 37. It will be understood, however, that the tubular arm member 37 could be inverted using this same technique without departing from the true spirit and nature of the present invention. It will further be appreciated that the inversion can occur either when the working element is in its initial cylindrical shell body 51 or in the final expanded shape of FIG. 9, as will be described.

To further radially expand the cylindrical shell body 51, forming the final working element shape of FIGS. 3 or 7 having the inwardly tapered proximal and distal ends, a mold shell 55 (FIG. 8) is employed. This mold shell 55 includes an interior cavity 57 having a shape substantially similar to the desired final shape of the working element 25 where the central bladder portion 26 has a diameter greater than the distal and proximal ends.

To accommodate for the tubular arm member 37 of the initial shape of the working element (i.e., cylindrical shell body 51), the mold shell 55 may include a strategically placed side port 56 that is sized and formed for receipt of the arm member therein. Once the cylindrical shell body 51 is placed in the cavity 57 of the mold shell 55, and the tubular arm member 37 is aligned with, and received in, the side port 56, the inflation passage 27 is pressurized. By heating the pressurized cylindrical shell body 51, the working element 25 will be permanently deformed from its initial molded shape into its operational shape of FIGS. 3 or 7. By way of example, for a balloon catheter element composed of nylon, it may be heated in a range of about 60° C. to about 120° C. with an internal pressure in the range of about 20 Bar to about 40 Bar. It will of course be appreciated that other heats and pressure combinations may apply.

Effectively, during the formation of the bladder portion 26, the internal pressurization of the working element causes the walls to expand and deform radially outwardly until the sidewall 31 of the bladder portion 26 contact the interior walls of the mold shell cavity 57. These deformed sidewalls 31 are uniformly thinned, compared to the walls of the proximal and distal portions, as they are deformed into a larger diameter. Subsequently, in this configuration, the bladder portion 26 or the tubular arm member 37 would then be inverted using one of the techniques above-mentioned.

Alternatively, the initially molded working element can be inverted first, and then deformed using the mold shell 55. In this embodiment, the side port 56 of the mold shell 55 will of course not be required since the inverted tubular arm member 37 will not require accommodation on the exterior of the working element. The second guidewire lumen 43 of the tubular arm member 37, however, will need to be blocked so as to enable pressurization of the inflation passage 27. This temporary blockage can be anywhere along the tubular arm member such as near the second end port 42.

Although the present invention has been described in connection with the preferred form of practicing it and modifications thereto, those of ordinary skill in the art will understand that many other modifications can be made thereto within the scope of the claims that follow. Accordingly, it is not intended that the scope of the invention in any way be limited by the above description, but instead be determined entirely by reference to the claims that follow.

What is claimed is:

1. A unitary inflatable working element for a catheter device suitable for treating a vessel bifurcation, said catheter device including an elongated flexible tubular shaft containing an inflation lumen therethrough, said working element comprising:
a hollow bladder device selectively inflatable from a first condition to an expanded second condition, said bladder device defining an inflation passage extending from a proximal end opening to a distal end opening thereof, said proximal end opening being configured to cooperate with a distal portion of the elongated shaft such said inflation passage is in access communication with the shaft inflation lumen; and
an tubular arm member configured to be disposed in the inflation passage, and having a first portion defining a first end port, accessible through the proximal end opening of the bladder device, and an opposed second portion, defining a second end port, and being coupled to, and terminating at, a sidewall of the bladder device in a fluid-tight manner.

2. The inflatable working element according to claim 1, wherein
said tubular arm member is integrally formed with the bladder device at said second end port.

3. The inflatable working element according to claim 2, wherein
said bladder device and said tubular arm member comprise a one-piece formed working element.

4. The inflatable working element according to claim 1, wherein said tubular arm is sized such that the first end port extends through and terminates proximal to the proximal end opening of the bladder device.

5. The inflatable working element according to claim 1, wherein
said tubular arm is sized such that the first end port terminates distal to the proximal end opening of the bladder device.

6. The inflatable working element according to claim 1, wherein
said tubular arm tapers radially inward from the second portion thereof toward the first portion thereof.

7. The inflatable working element according to claim 1, wherein
a longitudinal axis of said tubular arm, in a natural state, and the longitudinal axis of the bladder device are substantially contained in a same plane.

8. The inflatable working element according to claim 1, wherein
the longitudinal axis of said tubular arm at the second portion thereof, in the natural state, is oriented at an angle in the range of about 20° to about 90° relative to the longitudinal axis of the bladder device.

9. A method of fabricating an inflatable balloon element for a catheter device suitable for treating a vessel bifurcation, said method comprising:
forming a unitary balloon element having a hollow bladder portion defining an inflation passage extending from a proximal end opening to a distal end opening thereof, said body portion further including a flexible tubular arm member having a first end defining a first end port directed generally radially away from said body portion of the balloon element, and an opposed second end integrally formed in a sidewall of the body portion, said second end defining a second end port terminating at the body portion inflation passage such that said second end port is in direct access communication with the inflation passage; and
inverting one of said tubular arm member and said hollow bladder portion inside out such that said arm member is now disposed in the inflation passage, and having said first end port accessible through the proximal end opening of the bladder device and the opposed second end terminating at the sidewall in a manner such that said second end port is out of direct access communication with the inflation passage.

10. The method according to claim 9, wherein
said forming a unitary balloon element includes molding the hollow bladder portion about a balloon mold device as a one-piece element configured in a predetermined shape to form a shell body.

11. The method according to claim 9, wherein
said forming a unitary balloon element includes electro-grafting the hollow bladder portion and the arm member about a balloon mold device as a one-piece element configured in a predetermined shape to form a shell body.

12. The method according to claim 10, wherein
said forming a unitary balloon element includes molding the tubular arm member about a core pin removably mounted into a side of the first mold device.

13. The method according to claim 12, wherein
said forming a unitary balloon element further includes removing the core pin from the first mold device to form the tubular arm member.

14. The method according to claim 10, wherein
said forming a unitary balloon element further includes placing the shell body into a chamber of a mold shell having interior walls substantially preshaping the chamber into the desired final shape of the balloon element; and
applying heat to the chamber; and
inflating the inflation passage of the balloon element, expanding the hollow bladder against the interior walls of the mold shell.

15. The method according to claim 14, wherein
prior to applying heat, inserting the tubular arm into a side port of the interior walls of the mold shell formed and dimensioned for axial receipt of the tubular arm therein.

16. The method according to claim 9, wherein
said forming a unitary balloon element includes selecting a length of the tubular arm such that the first end port extends through and terminates proximal to the proximal end opening of the hollow bladder portion.

17. The method according to claim 9, wherein
said forming a unitary balloon element includes selecting a length of the tubular arm such that the first end port terminates distal to the proximal end opening of the hollow bladder portion.

18. A method of fabricating a catheter device suitable for treating a vessel bifurcation, said method comprising:
forming a unitary balloon element having a hollow bladder portion defining an inflation passage extending from a proximal end opening to a distal end opening thereof, said body portion further including a flexible tubular arm member having a first end defining a first end port directed generally radially away from said body portion of the balloon element, and an opposed second end integrally formed in a sidewall of the body portion, said second end defining a second end port terminating at the body portion inflation passage such that said second end port is in direct access communication with the inflation passage;
inverting one of said tubular arm member and said hollow bladder portion inside out such that said arm member is now disposed in the inflation passage, and having said first end port accessible through the proximal end opening of the bladder device and the opposed second end terminating at the sidewall in a manner such that said second end port is out of direct access communication with the inflation passage; and
mounting said proximal end of the balloon element to a distal portion of an elongated shaft of said catheter device such that said inflation passage of the balloon element is in flow communication with an inflation lumen of the catheter elongated shaft.

19. The method according to claim 18, wherein
said forming a unitary balloon element includes selecting a length of the tubular arm such that the first end port extends through and terminates proximal to the proximal end opening of the hollow bladder portion; and
said mounting includes positioning the first end of the tubular arm through the proximal end opening of the hollow bladder portion, and between the distal portion of the elongated shaft and the hollow bladder portion.

20. The method according to claim 9, wherein
said forming a unitary balloon element includes selecting a length of the tubular arm such that the first end port terminates distal to the proximal end opening of the hollow bladder portion; and
said mounting includes coupling the first end port of the tubular arm in fluid-tight access communication with a guidewire lumen extending through the elongated shaft.

21. A catheter device comprising:
an elongated flexible tubular shaft containing an inflation lumen and a main guidewire lumen therethrough;
an inflatable working element associated with the flexible tubular shaft, and including a hollow bladder portion selectively inflatable from a first condition to an expanded second condition, said bladder portion defining an inflation passage extending from a proximal end opening to a distal end opening thereof, said proximal end opening being configured to cooperate with a portion of the elongated shaft such said inflation passage is in access communication with the shaft inflation lumen, and a tubular arm member configured to be disposed in the inflation passage, and having a first portion defining a first end port accessible through the proximal end opening of the working element and an opposed second portion, defining a second end port, and being coupled to, and terminating at, a sidewall of the bladder portion in a fluid-tight manner.

22. The catheter device according to claim 21, wherein
said bladder device and said tubular arm member comprise a one-piece formed working element.

23. The catheter device according to claim 21, wherein
said tubular arm is sized such that the first end port extends through and terminates proximal to the proximal end opening of the bladder device such that the first end of the tubular arm extends through the proximal end opening of the hollow bladder portion, and between a distal portion of the elongated shaft and the hollow bladder portion.

24. The catheter device according to claim 1, wherein
said tubular arm is sized such that the first end port terminates distal to the proximal end opening of the bladder device within the inflation passage, and
said elongated shaft including a secondary guidewire lumen in fluid-tight access communication with first end port of the tubular arm.

25. The catheter device according to claim 24, wherein
said main guidewire lumen extends through a distal end of elongated shaft, the proximal end opening of the hollow bladder portion and the distal end opening thereof.

26. The catheter device according to claim 25, wherein
the secondary guidewire lumen and the main guidewire lumen are in access communication with one another.

27. The catheter device according to claim 21, wherein
the longitudinal axis of said tubular arm at the second portion thereof, in the natural state, is oriented at an angle in the range of about 20° to about 90° relative to the longitudinal axis of the bladder device.
